(19) United States
(12) Patent Application Publication
(10) Pub. No.: US 2024/0108691 A1
(43) Pub. Date: Apr. 4, 2024

(54) TREATMENT OF HEPATITIS E VIRUS INFECTION WITH INTERFERON LAMBDA

(71) Applicants: Eiger Biopharmaceuticals, Inc., Palo Alto, CA (US); Erasmus MC, Rotterdam (NL)

(72) Inventors: Andre Boonstra, Rotterdam (NL); Thomas Vanwolleghem, Rotterdam (NL); Gulce Sari, Rotterdam (NL)

(73) Assignees: Eiger Biopharmaceuticals, Inc., Palo Alto, CA (US); Erasmus MC, Rotterdam (NL)

(21) Appl. No.: 18/038,157

(22) PCT Filed: Nov. 30, 2021
(86) PCT No.: PCT/US2021/061155

§ 371 Date: May 22, 2023

Related U.S. Application Data
(60) Provisional application No. 63/119,524, filed on Nov. 30, 2020.
(60) Provisional application No. 63/149,788, filed on Feb. 16, 2021.

(51) Int. Cl. A61K 38/21; A61K 31/7056; A61K 47/60; A61P 31/14

(52) U.S. Cl. CPC A61K 38/21; A61K 31/7056; A61K 47/60; A61P 31/14

(57) ABSTRACT

Provided herein are methods of treating a hepatitis E virus (HEV) infection in a human patient are provided. The methods comprise administering to the patient a therapeutically effective amount of interferon lambda.

### CROSS-REFERENCE TO RELATED APPLICATIONS

This application claims the benefit of priority to U.S. Provisional Application No. 63/119,524, filed Nov. 30, 2020, and U.S. Provisional Application No. 63/149,788, filed Feb. 16, 2021, each of which are incorporated by reference in their entireties herein for all purposes.

### SEQUENCE LISTING

The official copy of the sequence listing is submitted electronically via EFS-Web as an ASCII formatted sequence listing with a file named 003810PC-SEQLISTING.txt, created Nov. 30, 2021, and having a size of 996 Bytes, and is filed concurrently with the specification. The sequence listing contained in this ASCII formatted document is part of the specification and is herein incorporated by reference in its entirety.

### FIELD

The present disclosure relates generally to methods for treating viral hepatitis resulting from hepatitis E virus (HEV) infection, and so relates to the fields of chemistry, medicinal chemistry, medicine, molecular biology, and pharmacology.

### BACKGROUND

Hepatitis E viruses (HEV) are an underestimated global cause of enterically transmitted viral hepatitis, which can persist in immunocompromised hosts. HEV infections are one of the leading causes of acute viral hepatitis and result in up to 70,000 deaths worldwide each year.

HEV is a non-enveloped single-stranded RNA (ssRNA) virus with a full length genome of ˜7.2 kb that belongs to the Orthohepevirus A species. Based on nucleotide divergence, 8 HEV genotypes (gt) have been identified, of which gt1, gt2, gt3, gt4, and gt7 have been reported to infect humans. Furthermore, humans are known to be the main reservoir of HEV gt1 and gt2, which are typically associated with waterborne epidemics in developing countries. Despite typically resulting in a mostly self-limiting acute hepatitis in humans, HEV gt3, gt4, and gt7 have a broad host range and can cause a chronic infection in patients with an immunocompromised status (e.g., a solid organ transplant, a hematopoietic malignancy or transplant, an autoimmune disease or a human immunodeficiency virus (HIV) infection.

Although no controlled clinical studies have determined the optimal treatment for HEV infection (e.g., chronic HEV infection), the conventional treatment options for (chronic) HEV infections are limited to ribavirin (RBV) or pegylated (peg) interferon alfa (IFN-α) and, for immunocompromised patient, reduction of immunosuppressive drugs. These treatment options are hampered by suboptimal antiviral efficacy as well as undesirable side effects, which may be severe. There is a pressing need for new options for treating HEV infection that demonstrate improved (e.g., increased) efficacy and/or reduced (e.g., eliminated) negative side effects.

### SUMMARY

Interferon lambda (IFN-λ) is a valuable alternative for the treatment of HEV infection. In particular, the preferential expression of the IFN-k receptor on hepatocytes and the less abundant presence on other cells (e.g., as compared to the IFN-α receptor) suggest that IFN-λ may be particularly well-suited to HEV treatment. The present disclosure describes several embodiments of treating HEV infections by administration of IFN-λ.

As detailed herein, the present inventors have carried out studies to assess the relative in vitro and in vivo potency for IFN-k (e.g., pegIFN-λ) to induce innate immune signaling in (human) liver cells and liver humanized mice. Furthermore, the present inventors have carried out studies to test the in vivo anti-HEV antiviral efficacy of various IFN-λ (e.g., pegIFN-λ) therapy durations and doses. In these studies, Type III IFN induction was observed after infection with the cell-culture adapted Kernow strain of HEV, but was not observed with a patient-derived strain belonging to the same HEV gt3 Glade. Primary hepatocytes and a hepatoma cell line were found to express the IFN-λ receptor and be responsive to pegIFN-λ, treatment in vitro. In vivo studies in liver humanized mice further revealed that pegIFN-λ, treatment can clear HEV gt3 infection. Although HEV gt3 antiviral efficacy of equimolar pegIFN-λ, was found to be lower than pegIFN-α, the present inventors have found that pegIFN-λ, treatment was well tolerated up to 10-fold higher doses (e.g., as compared to pegIFN-α).

Thus, the present inventors have determined that IFN-λ (e.g., pegIFN-λ) is a valuable therapeutic agent for the treatment of HEV infections (e.g., chronic HEV infections, such as chronic HEV gt3 infections).

In some cases, the present disclosure provides methods of treating a hepatitis E virus (HEV) infection in a human patient, the method comprising administering the patient a therapeutically effective amount of interferon lambda.

### BRIEF DESCRIPTION OF THE DRAWINGS

The disclosure is described in detail below with reference to the appended drawings.

FIGS. 1A-1B show the induction of IFN-λ3 mRNA expression in vivo between HEV strains, and particularly that the induction varies between strains, according to aspects of this disclosure. FIG. 1A shows relative IFN-λ1, IFN-λ2 and IFN-λ3 mRNA expression levels of HEV gt3c infected and non-infected mice, which were measured using a qPCR method. IFN-λ-3 expression was detected following pegIFN-α treatment, irrespective of the infection status. FIG. 1B shows tests from Example 1, wherein human liver chimeric mice were infected with either clinical strain HEV gt3c (gt3c) or with HEV Kernow strain (K) for 6 weeks. IFN-λ3 mRNA induction was obtained from Klenow strain infected mice liver after 6 weeks of infection. Each dot represents an individual mouse. Uninfected mice were controls. Lines show Mean±SE. Primary hepatocytes donor details are provided in Table 1, and experimental group details are provided in Table 4.

FIGS. 2A-2B show that primary human hepatocytes (PHH) are responsive to exogenous IFN-λ, treatment in vitro, according to aspects of this disclosure. FIG. 2A shows IFN-λ, receptor, IFN-λR1, relative mRNA expression levels of primary hepatocytes (donor342) after either 0.01 mg/L pegIFN-α or 0.1 mg/mL pegIFN-λ, treatments for 1 to 5 hours. Each dot represents the average of three independent replicates. FIG. 2B shows interferon stimulated gene (ISG) mRNA induction of human hepatoma cell line (HepG2 cells, circled dots) and primary human hepatocytes (donor342) following either 0.01 mg/L pegIFN-α or 0.1 mg/mL pegIFN-λ, or with PBS treatment for 5 hours. Each dot shows a biological replicate and lines show Mean±SE, *p<0.05, **p<0.01, ***p<0.001. In the IFIT1 graph, the data for the Medium controls for both HepG2 and PHH cells are overlapping at baseline, thus the circle encompasses all the data points.

FIGS. 3A-3D show that pegIFN-λ, treatment clears HEV infection in liver humanized mouse model, according to aspects of this disclosure. FIG. 3A shows HEV RNA titers in fecal (left), bile and liver (right) of a liver humanized mouse model infected with HEV gt3c. After 6 weeks of infection, mice received a single subcutaneous (sc) injection of 0.03 mg/kg pegIFN-λ, twice weekly (every 3-4 days), for 2 weeks. FIG. 3B shows the titration of pegIFN-λ dose. HEV infected mice received a range of pegIFN-λ doses up to 0.3 mg/kg for up to 8 weeks. Titration details are provided in Table 5. Each dot represents an individual mouse, and lines show Mean±SE. FIG. 3C shows treatment with curative dose of pegIFN-λ in liver humanized mouse model infected with HEV gt3c. After confirming the successful infection, mice received a single sc 0.3 mg/kg pegIFN-λ (n=7) or sc 0.03 mg/kg pegIFN-α (n=4) injections, every 3-4 days, for 2 weeks. Other mice left untreated (n=2) as infection controls. Each dot represents an individual mouse. FIG. 3D shows confocal images of liver sections stained for human albumin (anti-human albumin, A80-129A) and HEV viral antigens (anti-ORF2, chimpanzee 1313 serum). Scale bar: 10 μm.

FIG. 4A and FIG. 4B show IFN-λR1 expression and ISG stimulation, respectively, following 2 weeks of pegIFN-λ or pegIFN-α treatment in vivo, according to aspects of this disclosure. In both cases, non-infected (n=2) and HEV gt3c infected mice received a single sc 0.3 mg/kg pegIFN-λ injection (n=4), 0.03 mg/kg pegIFN-λ (n=2), or a single sc 0.03 mg/kg pegIFN-α injection (n=4), every 3-4 days for 2 weeks. Treatment naïve, non-infected (n=4) and HEV gt3c infected (n=3) mice were included as controls. Each dot represents an individual mouse; square: mice transplanted with PHH Donor2 and circle: mice transplanted with PHH Donor3. Lines show Mean±SE. Taqman probe details of ISGs are provided in Table 3A.

FIG. 4C shows a heatmap of ISG expression from the same animals as described for FIGS. 4A-4B, according to aspects of this disclosure. The color scale bar indicates the relative mRNA expression level in comparison to non-treated animals (n/a) and non-infected animals (HEV−). Darker shading represents a significant increase, and white-light shading represent a very low expression.

### DETAILED DESCRIPTION

### I. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

Although any methods and materials similar or equivalent to those described herein can be used in the practice or testing of the present invention, the preferred methods, devices, and materials are now described. All patents, patent publications, patent applications, journal articles, books, technical references, and the like discussed in the instant disclosure are incorporated herein by reference in their entirety for all purposes. Nothing herein is to be construed as an admission that the invention is not entitled to antedate such disclosure by virtue of prior invention.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “administration” refers to introducing a compound, a composition, or an agent of the present disclosure into a host, such as a human. One preferred route of administration of the agents is subcutaneous administration. Other routes are intravenous administration and oral administration.

The term “baseline,” unless otherwise specified or apparent from context, refers to a measurement (of, e.g., viral load, patient condition, ALT level) made prior to a course of therapy.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

The terms “course of treatment” and “course of therapy” are used interchangeably herein, and refer to the medical interventions made after a patient is diagnosed, e.g., as being infected with HEV and in need of medical intervention. Medical interventions include, without limitation, the administration of drugs for a period of time, typically, for HEV infected patients, at least one and typically several or many months or even years.

The term “HEV RNA viral load” or “viral load” of a biological sample (e.g., a human serum or plasma sample) refers to the amount of HEV RNA in a given amount of the biological sample. HEV RNA is generally detected by quantitative real-time reverse transcription-polymerase chain reaction (qRT-PCR) assays. In such assays, the amount of signal generated during the assay is proportional to the amount of HEV RNA in the sample. The signal from the test sample is compared to that of a dilution series of a quantified standards, and a copy number of genome copies is calculated. See, e.g., van de Garde et al., Hepatitis E Virus (HEV) Genotype 3 Infection of Human Liver Chimeric Mice as a Model for Chronic HEV Infection, J. Virol., 2016. 90(9): p. 4394-4401; Pas et al., Hepatitis E virus infection among solid organ transplant recipients, the Netherlands, Emerg. Infect. Dis, 2012. 18(5): p. 869-872. The qRT-PCR may be validated according to IS015189:2012.

HEV RNA viral load may be reported as RNA copies per mL serum (or plasma) or using International Units (IU) per mL serum (or plasma). HEV RNA viral load may also be reported as RNA copies per mg tissue or using International Units (IU) per mg tissue. Unless otherwise specified, reference to below the level of detection means below 100 IU/g.

HEV levels are generally presented using log 10 units, following the normal conventions of virology.

Changes in HEV RNA levels may be represented as a “log reduction” following the normal conventions of virology. For example, a 1 log reduction (i.e., −1 log) in viral load (e.g., from 7 log to 6 log) is a 10-fold reduction, and a 2 log reduction (i.e., −2 log) in viral load (e.g., from 7 log to 5 log) is a 100-fold reduction. A reduction from 4 log RNA copies/mL to 3 log RNA copies/mL is equivalent to a reduction from 4 log IU/mL to 3 log IU/mL.

The term “HEV infection” with respect to a host subject (e.g., human) refers to the fact that the host is suffering from HEV infection. Typically, an HEV infected human host will have a viral load of HEV RNA of at least about 2 log HEV RNA copies/mL of host serum or plasma or 10² copies of REV-RNA/mL of host serum or plasma, often at least about 3 log HEV RNA copies/mL of host serum or plasma or 10³ copies of REV-RNA/mL of host serum or plasma, and, often, especially for patients not on any therapy, at least about 4 log HEV RNA copies/mL of host serum or plasma or 10⁴ copies of REV-RNA/mL of host serum or plasma, such as about 4 log HEV RNA copies/mL of host serum or plasma to 8 log HEV RNA copies/mL of host serum or plasma or 10⁴-10⁸ copies of REV-RNA/mL of host serum or plasma.

As used herein, the term “chronic HEV infection” with respect to a human host refers to an HEV infection that has persisted in the human host for at least 6 months, as documented by a positive REV antibody (Ab) test and/or detectable HEV RNA by qRT-PCR.

The terms “patient”, “host,” or “subject,” are used interchangeably and refer to a human infected with HEV, including patients previously infected with HEV in whom virus has cleared.

The term “pharmaceutical composition” is meant to encompass a composition suitable for administration to a subject. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intratracheal, intramuscular, subcutaneous, inhalational, and the like.

The term “therapeutically effective amount” as used herein refers to that amount of an embodiment of the agent (e.g., a compound, inhibitory agent, or drug) being administered that will treat to some extent a disease, disorder, or condition, e.g., relieve one or more of the symptoms of the disease, i.e., infection, being treated, and/or that amount that will prevent, to some extent, one or more of the symptoms of the disease, i.e., infection, that the subject being treated has or is at risk of developing.

The terms “treatment”, “treating”, and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a human subject, and includes: (a) reducing the risk of occurrence of the disease in a subject determined to be predisposed to the disease but not yet diagnosed as infected with the disease, (b) impeding the development of the disease, and/or (c) relieving the disease, i.e., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of an agent that provides for enhanced or desirable effects in the subject (e.g., reduction of viral load, reduction of disease symptoms, etc.).

The terms “undetectable” or “below the level of detection,” as used with reference to HEV RNA levels, means that no HEV RNA copies can be detected by the assay methodology employed. In some embodiments, the assay is quantitative RT-PCR (qRT-PCR).

### II. Methods of Treatment

In some aspects, the present disclosure provides methods for the treatment of HEV infection, in which the HEV-infected patient is treated by administration of interferon lambda. In some embodiments, a pegylated form of interferon lambda is administered. In some embodiments, patients receiving interferon lambda therapy (e.g., pegylated interferon lambda therapy) are also treated with the antiviral nucleotide or nucleoside analog (e.g., ribavirin).

The present inventors identified, for the first time, in vivo antiviral efficacy of IFN-λ, (e.g., pegIFN-λ) as a new therapeutic for treatment of chronic HEV infections using a liver humanized mouse model. Both IFN-α and IFN-λ, show antiviral, anti-proliferative and immunomodulatory effects, and both cytokines are potent inducers of JAK/STAT pathway, initiated after their engagement with their respective receptors. Differently, ISG induction and antiviral activity by IFN-λ, treatment are delayed and milder compared to IFN-α. One surprising and unexpected result determined by the inventors is that, although IFN-λ, and IFN-α stimulate the same signaling cascade, at the same dose, IFN-λ, was not able to clear the HEV infection after 8 weeks of treatment while IFN-α resulted in full sterilization after 2 weeks of treatment. Dose escalation studies showed that although HEV was not cleared at pegIFN-λ, doses up to 0.12 mg/kg for a maximum of 8 weeks, a dose of 0.3 mg/kg pegIFN-λ, treatment resulted in complete clearance of HEV antigen and HEV RNA from the liver in 8 out of 9 liver-humanized mice. The inventors also demonstrated that signal initiation by IFN-λ, receptors is 10 times less potent than IFN-α signal initiation both in vivo and in vitro. Thus, IFN-λ, and IFN-α have different potencies and different timing of responses in vivo. Despite this, IFN-λ, treatment was well tolerated up to 10-fold higher doses as compared to IFN-α. In particular, the inventors have demonstrated that IFN-λ, (e.g., pegIFN-λ) is particularly advantageous because fewer side effects are expected and liver humanized animals were able to tolerate even high doses. As described herein, treatment with pegIFNλ, of liver-humanized mice persistently infected with HEV genotype 3 showed that pegIFNλ, was well tolerated. This is in contrast to the severe side effects demonstrated in pegIFN-α treatment.

### Interferon Lambda Therapy

Interferons (IFNs) are polypeptides that inhibit viral replication and cellular proliferation and modulate immune response. Based on the type of receptor through which they signal, human interferons have been classified into three major types (Types I, II, and III). All type I IFNs bind to a specific cell surface receptor complex known as the IFN-alpha receptor (IFNAR) that consists of IFNAR1 and IFNAR2 chains. The type I interferons present in humans are IFN-alpha, IFN-beta, IFN-epsilon, and IFN-omega. Type II IFNs bind to IFN-gamma receptor (IFNGR) that consists of IFNGR1 and IFNGR2 chains. The type II interferon in humans is IFN-gamma. The type III interferon group consists of three IFN-lambda molecules called IFN-lambda1, IFN-lambda2 and IFN-lambda3 (also called IL29, IL28A, and IL28B, respectively). These IFNs signal through a receptor complex consisting of IL10R2 (also called CRF2-4) and IFNLR1 (also called CRF2-12).

The term “interferon-lambda” or “IFN-λ,” as used herein includes naturally occurring IFN-λ; synthetic IFN-λ; derivatized IFN-λ, (e.g., PEGylated glycosylated IFN-λ, and the like); and analogs of naturally occurring or synthetic IFN-λ. In some embodiments, an IFN-λ, is a derivative of IFN-λ, that is derivatized (e.g., chemically modified relative to the naturally occurring peptide) to alter certain properties such as serum half-life. As such, the term “IFN-λ,” includes IFN-λ, derivatized with polyethylene glycol (“PEGylated IFN-λ”), and the like. PEGylated IFN-λ, (e.g., PEGylated IFN-λ-1a), and methods for making same, is discussed in, e.g., U.S. Pat. Nos. 6,927,040; 7,038,032; 7,135,170; 7,157,559; and 8,980,245; and PCT Publication Nos. WO 05/097165, WO 07/012,033, WO 07/013,944, and WO 07/041,713; all of which are herein incorporated by reference in their entirety.

In some embodiments, an interferon for use in a therapeutic method as described herein is a pegylated IFN-λ-1 (e.g., pegylated IFN-λ-1a), pegylated IFN-λ-2, or pegylated 3.

The dose of interferon lambda administered to the patient is not particularly limited. As detailed below, the present inventors have found that relatively high doses of interferon lambda are tolerated. Thus, in some cases, the interferon lambda is administered at a relatively high dose. In some embodiments, the interferon lambda is administered at a dose from 0.5 mg to 500 mg, e.g., from 1 mg to 500 mg, 1.5 mg to 500 mg, from 2 mg to 500 mg, 2.5 mg to 500 mg, from 3 mg to 500 mg, from 3.5 mg to 500 mg, from 4 mg to 500 mg, from 4.5 mg to 500 mg, from 5 mg to 500 mg, from 0.5 mg to 450 mg, from 1 mg to 450 mg, 1.5 mg to 450 mg, from 2 mg to 450 mg, 2.5 mg to 450 mg, from 3 mg to 450 mg, from 3.5 mg to 450 mg, from 4 mg to 450 mg, from 4.5 mg to 450 mg, from 5 mg to 450 mg, from 0.5 mg to 400 mg, from 1 mg to 400 mg, 1.5 mg to 400 mg, from 2 mg to 400 mg, 2.5 mg to 400 mg, from 3 mg to 400 mg, from 3.5 mg to 400 mg, from 4 mg to 400 mg, from 4.5 mg to 400 mg, from 5 mg to 400 mg, from 0.5 mg to 350 mg, from 1 mg to 350 mg, 1.5 mg to 350 mg, from 2 mg to 350 mg, 2.5 mg to 350 mg, from 3 mg to 350 mg, from 3.5 mg to 350 mg, from 4 mg to 350 mg, from 4.5 mg to 350 mg, from 5 mg to 350 mg, from 0.5 mg to 300 mg, from 1 mg to 300 mg, 1.5 mg to 300 mg, from 2 mg to 300 mg, 2.5 mg to 300 mg, from 3 mg to 300 mg, from 3.5 mg to 300 mg, from 4 mg to 300 mg, from 4.5 mg to 300 mg, from 5 mg to 300 mg, from 0.5 mg to 250 mg, from 1 mg to 250 mg, 1.5 mg to 250 mg, from 2 mg to 250 mg, 2.5 mg to 250 mg, from 3 mg to 250 mg, from 3.5 mg to 250 mg, from 4 mg to 250 mg, from 4.5 mg to 250 mg, from 5 mg to 250 mg, from 0.5 mg to 200 mg, from 1 mg to 200 mg, 1.5 mg to 200 mg, from 2 mg to 200 mg, 2.5 mg to 200 mg, from 3 mg to 200 mg, from 3.5 mg to 200 mg, from 4 mg to 200 mg, from 4.5 mg to 200 mg, from 5 mg to 200 mg, from 0.5 mg to 150 mg, from 1 mg to 150 mg, 1.5 mg to 150 mg, from 2 mg to 150 mg, 2.5 mg to 150 mg, from 3 mg to 150 mg, from 3.5 mg to 150 mg, from 4 mg to 150 mg, from 4.5 mg to 150 mg, from 5 mg to 150 mg, from 0.5 mg to 100 mg, from 1 mg to 100 mg, 1.5 mg to 100 mg, from 2 mg to 100 mg, 2.5 mg to 100 mg, from 3 mg to 100 mg, from 3.5 mg to 100 mg, from 4 mg to 100 mg, from 4.5 mg to 100 mg, from 5 mg to 100 mg, from 0.5 mg to 50 mg, from 1 mg to 50 mg, 1.5 mg to 50 mg, from 2 mg to 50 mg, 2.5 mg to 50 mg, from 3 mg to 50 mg, from 3.5 mg to 50 mg, from 4 mg to 50 mg, from 4.5 mg to 50 mg, from 5 mg to 50 mg, from 0.5 mg to 25 mg, from 1 mg to 25 mg, 1.5 mg to 25 mg, from 2 mg to 25, 2.5 mg to 25 mg, from 3 mg to 25 mg, from 3.5 mg to 25 mg, from 4 mg to 25 mg, from 4.5 mg to 25 mg, or from 5 mg to 25 mg.

In some embodiments, the interferon lambda is administered at a dose from 5 mg to 30 mg, e.g., from 5 mg to 28 mg, from 5 mg to 26 mg, from 5 mg to 24 mg, from 5 mg to 22 mg, from 5 mg to 20 mg, from 8 mg to 30 mg, from 8 mg to 28 mg, from 8 mg to 26 mg, from 8 mg to 24 mg, from 8 mg to 22 mg, from 8 mg to 20 mg, from 10 mg to 30 mg, from 10 mg to 28 mg, from 10 mg to 26 mg, from 10 mg to 24 mg, from 10 mg to 22 mg, from 10 mg to 20 mg, from 12 mg to 30 mg, from 12 mg to 28 mg, from 12 mg to 26 mg, from 12 mg to 24 mg, from 12 mg to 22 mg, from 12 mg to 20 mg, from 15 mg to 30 mg, from 15 mg to 28 mg, from 15 mg to 26 mg, from 15 mg to 24 mg, from 15 mg to 22 mg, or from 15 mg to 20 mg. In terms of lower limits, the interferon lambda may be administered at a dose greater than 5 mg, e.g., greater than 8 mg, greater than 10 mg, greater than 12 mg, or greater than 15 mg. In terms of upper limits, the interferon lambda may be administered at a dose less than 30 mg, e.g., less than 28 mg, less than 26 mg, less than 24 mg, less than 22 mg, or less than 20 mg. In some cases, the interferon lambda is administered at a dose of 18 mg.

In some cases, the interferon lambda is administered at a relatively low dose. In some embodiments, the interferon lambda is administered at a dose from 1 μg to 500 from 2 μg to 500 from 3 μg to 500 from 4 μg to 500 from 5 μg to 500 from 1 μg to 450 μg, from 2 μg to 450 μg, from 3 μg to 450 μg, from 4 μg to 450 μg, from 5 μg to 450 μg, from 1 μg to 400 μg, from 2 μg to 400 μg, from 3 μg to 400 μg, from 4 μg to 400 μg, from 5 μg to 400 μg, from 1 μg to 350 μg, from 2 μg to 350 μg, from 3 μg to 350 μg, from 4 μg to 350 μg, from 5 μg to 350 μg, from 1 μg to 300 μg, from 2 μg to 300 μg, from 3 μg to 300 μg, from 4 μg to 300 μg, from 5 μg to 300 μg, from 1 μg to 250 μg, from 2 μg to 250 μg, from 3 μg to 250 μg, from 4 μg to 250 μg, from 5 μg to 250 μg, from 1 μg to 200 μg, from 2 μg to 200 μg, from 3 μg to 200 μg, from 4 μg to 200 μg, from 5 μg to 200 μg, from 1 μg to 150 μg, from 2 μg to 150 μg, from 3 μg to 150 μg, from 4 μg to 150 μg, from 5 μg to 150 μg, from 1 μg to 100 μg, from 2 μg to 100 μg, from 3 μg to 100 μg, from 4 μg to 100 μg, from 5 μg to 100 μg, from 1 μg to 50 μg, 1.5 μg to 50 μg, from 2 μg to 50 μg, from 3 μg to 50 μg, from 4 μg to 50 μg, from 5 μg to 50 μg, from 1 μg to 25 μg, 1.5 μg to 25 μg, from 2 μg to 25, from 3 μg to 25 μg, from 4 μg to 25 μg, or from 5 μg to 25 μg. In some instances, the doses listed in the paragraph may be appropriate for administration to a mouse and doses appropriate for a human are scalable as described in Services USDoHaH, Administration FaD, (CDER) CfDEaR. Guidance for Industry Estimating the Maximum Safe Starting Dose in Initial Clinical Trials for Therapeutics in Adult Healthy Volunteers, U.S. Dept. of Health and Human Services, Food and Drug Administration, Center for Drug Evaluation and Research, Pharmacology and Toxicology, July 2005, available at www.fda.gov/media/72309/download.

In some cases, the dose is determined by the bodyweight of the patient (e.g., at the time of administration and/or at the time of the beginning of the treatment). In some embodiments, for example, the interferon lambda is administered at a dose from 10 μg/kg to 500 μg/kg, e.g., from 15 μg/kg to 500 μg/kg, 20 μg/kg to 500 μg/kg, 25 μg/kg to 500 μg/kg, 30 μg/kg to 500 μg/kg, 35 μg/kg to 500 μg/kg, 40 μg/kg to 500 μg/kg, 45 μg/kg to 500 μg/kg, 50 μg/kg to 500 μg/kg, from 10 μg/kg to 450 μg/kg, from 15 μg/kg to 450 μg/kg, 20 μg/kg to 450 μg/kg, 25 μg/kg to 450 μg/kg, 30 μg/kg to 450 μg/kg, 35 μg/kg to 450 μg/kg, 40 μg/kg to 450 μg/kg, 45 μg/kg to 450 μg/kg, 50 μg/kg to 450 μg/kg, from 10 μg/kg to 400 μg/kg, from 15 μg/kg to 400 μg/kg, 20 μg/kg to 400 μg/kg, 25 μg/kg to 400 μg/kg, 30 μg/kg to 400 μg/kg, 35 μg/kg to 400 μg/kg, 40 μg/kg to 400 μg/kg, 45 μg/kg to 400 μg/kg, 50 μg/kg to 400 μg/kg, from 10 μg/kg to 350 μg/kg, from 15 μg/kg to 350 μg/kg, 20 μg/kg to 350 μg/kg, 25 μg/kg to 350 μg/kg, 30 μg/kg to 350 μg/kg, 35 μg/kg to 350 μg/kg, 40 μg/kg to 350 μg/kg, 45 μg/kg to 350 μg/kg, 50 μg/kg to 350 μg/kg, from 10 μg/kg to 250 μg/kg, from 15 μg/kg to 250 μg/kg, 20 μg/kg to 250 μg/kg, 25 μg/kg to 250 μg/kg, 30 μg/kg to 250 μg/kg, 35 μg/kg to 250 μg/kg, 40 μg/kg to 250 μg/kg, 45 μg/kg to 250 μg/kg, or 50 μg/kg to 250 μg/kg. In some instances, the interferon lambda may be administered at a dose of 250-350 μg/kg or 275 μg/kg to 325 μg/kg.

In terms of lower limits, the interferon lambda may be administered at a dose greater than 10 μg/kg, e.g., greater than 15 μg/kg, greater than 20 μg/kg, greater than 25 μg/kg, greater than 30 μg/kg, greater than 35 μg/kg, greater than 40 μg/kg, greater 45 μg/kg, or greater than 50 μg/kg. In terms of upper limits, the interferon lambda may be administered at a dose less than 500 μg/kg, e.g., less than 450 μg/kg, less than 425 μg/kg, less than 400 μg/kg, less than 350 μg/kg, less than 300 μg/kg, or less than 275 μg/kg.

In some embodiments, the interferon lambda may be administered at a relatively low dose. For example, the interferon lambda may be administered at a dose from 10 μg/kg to 50 μg/kg, e.g., 15 μg/kg to 50 μg/kg, 20 μg/kg to 30 μg/kg, 20 μg/kg to 50 μg/kg, 25 μg/kg to 50 μg/kg, 30 μg/kg to 50 μg/kg, 10 μg/kg to 45 μg/kg, 15 μg/kg to 45 μg/kg, 20 μg/kg to 45 μg/kg, 25 μg/kg to 45 μg/kg, 30 μg/kg to 45 μg/kg, 10 μg/kg to 40 μg/kg, 15 μg/kg to 40 μg/kg, 20 μg/kg to 40 μg/kg, 25 μg/kg to 40 μg/kg, 30 μg/kg to 40 μg/kg, 10 μg/kg to 35 μg/kg, 15 μg/kg to 35 μg/kg, 20 μg/kg to 35 μg/kg, 25 μg/kg to 35 μg/kg, 30 μg/kg to 35 μg/kg. In some instances, the interferon lambda may be administered at a dose of 20-30 μg/kg.

In some embodiments, the interferon lambda may be administered at a relatively high dose. For example, the interferon lambda may be administered at a dose from 100 μg/kg to 500 μg/kg, e.g., 150 μg/kg to 500 μg/kg, 200 μg/kg to 500 μg/kg, 250 μg/kg to 500 μg/kg, 300 μg/kg to 500 μg/kg, 100 μg/kg to 450 μg/kg, 150 μg/kg to 450 μg/kg, 200 μg/kg to 450 μg/kg, 250 μg/kg to 450 μg/kg, 300 μg/kg to 450 μg/kg, 100 μg/kg to 400 μg/kg, 150 μg/kg to 400 μg/kg, 200 μg/kg to 400 μg/kg, 250 μg/kg to 400 μg/kg, 300 μg/kg to 400 μg/kg, 100 μg/kg to 350 μg/kg, 150 μg/kg to 350 μg/kg, 200 μg/kg to 350 μg/kg, 250 μg/kg to 350 μg/kg, 300 μg/kg to 350 μg/kg, or 275 μg/kg to 325 μg/kg. In some instances, the interferon lambda may be administered at a dose of 250-350 μg/kg or 275 μg/kg to 325 μg/kg. In some instances, the interferon lambda may be administered at a dose of 300 μg/kg±24.4 μg/kg.

In some embodiments, the patient is administered a single dose of interferon lambda. In some embodiments, the patient is administered multiple doses of interferon lambda (e.g., any of the above-described doses), which may be administered at predetermined intervals. In some cases, for example, the interferon lambda is administered once daily (QD), twice daily (BID), three times daily (TID), weekly (QW), and/or twice weekly (BW).

In some embodiments, the interferon lambda is administered (e.g., subcutaneously) at a dose of 30 μg/kg twice weekly, e.g., for 2 weeks, for 4 weeks, or for 8 weeks.

In some embodiments, the interferon lambda is administered (e.g., subcutaneously) at a dose of 60 μg/kg twice weekly, e.g., for 4 weeks.

In some embodiments, the interferon lambda is administered (e.g., subcutaneously) at a dose of 120 μg/kg twice weekly, e.g., for 4 weeks.

In some embodiments, the interferon lambda is administered (e.g., subcutaneously) at a dose of 300 μg/kg twice weekly, e.g., for 2 weeks. In some embodiments, the interferon lambda is administered (e.g., subcutaneously) at a dose of 300 μg/kg±24.4 μg/kg twice weekly, e.g., for 2 weeks.

### Patient Population

In some embodiments, a patient to be treated with interferon lambda therapy as described herein is a patient having an HEV infection. In some cases, for example, the patent to be treated with interferon lambda therapy has a chronic HEV infection. In one embodiment, for example, the patient to be treated has a chronic HEV infection defined as being viraemic for more than three months after HEV infection as may be determined, for example, by a positive HEV antibody (Ab) test and/or detectable HEV RNA by qRT-PCR. See, e.g., EASL Clinical Practice Guidelines on hepatitis E virus infection, J Hepatology, 68(6):1256-1271, June 2018, (doi: 10.1016/j.jhep.2018.03.005). In some embodiments, the patient to be treated has a chronic HEV infection of at least 6 months duration documented by a positive HEV antibody (Ab) test and/or detectable HEV RNA by qRT-PCR. In some embodiments, a patient to be treated with a therapeutic method described herein is a patient having an acute HEV infection, one that is newly diagnosed or otherwise believed not to have existed in the patient for more than six months. HEV is known to exist in a variety of subtypes (e.g., genotypes); the methods described herein are suitable for treating all HEV patients, regardless of HEV subtype. In some embodiments, the patient is an adult (18 years or older).

In some embodiments, a patient to be treated has a baseline viral load of at least 10⁴ HEV RNA copies per mL serum, e.g., at least 10⁵ HEV RNA copies per mL serum or plasma, at least 10⁶ HEV RNA copies per mL serum or plasma, at least 10⁷ HEV RNA copies per mL serum or plasma, or at least 10⁸ HEV RNA copies per mL serum or plasma. In some embodiments, the patient to be treated has a baseline viral load of at least 10⁶ HEV RNA copies per mL serum. In some embodiments, HEV viral load is measured using serum samples from the patient. In some embodiments, HEV viral load is measured using plasma samples from the patient. In some embodiments, HEV viral load is measured using tissue samples from the patient. In some embodiments, viral load is measured by quantitative RT-PCR (qRT-PCR). qRT-PCR assays for quantification of HEV RNA in serum or plasma are known in the art, e.g., as described above.

In some embodiments, a patient to be treated exhibits one or more symptoms of liver dysfunction. In some embodiments, the patient exhibits one or more liver function parameters that are outside the normal parameters for a healthy control (e.g., a subject that is not infected with HEV). In some embodiments, the liver function parameter is selected from the group consisting of serum albumin, bilirubin, alanine aminotransferase (ALT), aspartate aminotransferase (AST), and prothrombin activity. In some embodiments, the patient has a serum ALT level that is at least two-fold higher than the upper limit of normal (ULN) (e.g., at least 2-fold, at least 3-fold, at least 4-fold, at least 5-fold, at least 6-fold, at least 7-fold, at least 8-fold, at least 10-fold or higher than the ULN). Liver function parameters are described in the art. See, e.g., Limdi et al., Postgrad Med J, 2003, 79:307-312. Methods of measuring these liver function parameters are known in the art and are also commercially available.

In some embodiments, the patient has compensated liver disease (e.g., as classified according to the Child-Turcotte-Pugh Classification System) with or without liver cirrhosis. It will be recognized by a person of ordinary skill in the art that the Child-Turcotte-Pugh Classification System is used to classify the severity of liver disease and is determined by assessing serum albumin levels, bilirubin levels, international normalized ratio of prothrombin time levels, ascites formation, and encephalopathy. In some embodiments, the patient has a Child-Turcotte-Pugh score of 5-6 (class A). In some embodiments, the patient has compensated liver disease with liver cirrhosis. In some embodiments, the patient has compensated liver disease without liver cirrhosis.

In some embodiments, the patient is diagnosed with chronic hepatitis as determined by liver biopsy within 6 months before treatment. In some embodiments, the patient has evidence of chronic hepatitis based on a liver biopsy within 6 months before screening. In some embodiments, the patient has a serum alanine aminotransferase (ALT) level that is above the upper limit of normal (ULN) within 24 weeks prior to treatment and/or at the initiation of treatment.

### Duration of Treatment and Treatment Endpoints

Patients may receive interferon lambda therapy for a predetermined time, an indefinite time, or until an endpoint is reached. Treatment may be continued on a continuous daily basis for at least two to three months. In some embodiments, therapy is for at least 7 days, at least 10 days, at least 14 days, at least 20 days, at least 25 days, at least 30 days, at least 60 days, at least 90 days, at least 120 days, at least 150 days, or at least 180 days. In some embodiments, treatment is continued for at least 6 months, at least 7 months, at least 8 months, at least 9 months, at least 10 months, at least 11 months, at least one year, at least 15 months, at least 18 months, or at least 2 years. In some embodiments, therapy is for at least 1 week, at least 2 weeks, at least 4 weeks, at least 6 weeks, at least 8 weeks, 12 weeks, 18 weeks, 24 weeks, 30 weeks, 36 weeks, 42 weeks, 48 weeks, 60 weeks, 72 weeks, 84 weeks, or 96 weeks. In other embodiments, treatment is continued for the rest of the patient's life or until administration is no longer effective in maintaining the virus at a sufficiently low level to provide meaningful therapeutic benefit.

In some embodiments, treatment is continued until clearance of HEV. That is, some HEV patients will respond to therapy as described herein by clearing virus to undetectable levels, after which treatment may be suspended unless and until the HEV levels return to detectable levels. Other patients will experience a reduction in viral load and improvement of symptoms but will not clear the virus to undetectable levels but will remain on “long term therapy” for a defined period of time (e.g., for about 1 year or for about 2 years) or so long as it provides therapeutic benefit.

In some embodiments, treatment with interferon lambda therapy results in a reduction of HEV viral load in the patient of at least 1.5 log, e.g., at least 2.0 log, at least 2.5 log, at least 3.0 log, at least 3.5 log, at least 4.0 log, at least 4.5 log, or at least 5.0 log. In some embodiments, for examples, the treatment described herein results in an HEV viral load reduction of at least 1.5 log HEV RNA copies/mL serum, when measured after 4 weeks of treatment. In some embodiments, treatment with interferon lambda therapy results in a reduction of HEV viral load in the patient of at least 2.0 log HEV RNA copies/mL serum, when measured after 4 weeks of treatment. In some embodiments, treatment with interferon lambda therapy results in a reduction of HEV viral load in the patient of at least 2.5 log HEV RNA copies/mL serum, when measured after 4 weeks of treatment.

In some embodiments, treatment with interferon lambda therapy results in a sustained reduction of HEV viral load (e.g., a decrease of at least 1.5 log HEV RNA copies/mL serum, at least 2.0 log HEV RNA copies/mL serum or at least 2.5 log HEV RNA copies/mL serum, or a decrease in HEV RNA to undetectable levels) that is sustained for a period of time (e.g., 1 month, 3 months, 6 months, 1 year or longer) while the course of treatment is still ongoing. In some embodiments, treatment with interferon lambda therapy results in a sustained reduction of HEV viral load that is sustained for a period of time (e.g., 1 month, 3 months, 6 months, 1 year or longer) after the course of treatment is finished. In some embodiments, the course of treatment results in HEV RNA levels (e.g., serum HEV RNA levels or plasma HEV RNA levels) below 1,000 copies/mL. In some embodiments, the HEV RNA levels remain below 1,000 copies/mL for at least one month, at least three months, at least one year, or longer. In some embodiments, the course of treatment results in HEV RNA levels (e.g., serum HEV RNA levels or plasma HEV RNA levels) below 100 copies/mL. In some embodiments, the HEV RNA levels remain below 100 copies/mL for at least one month, at least three months, at least six months at least one year, or longer. The phrase “remains below” an initial measured value (e.g., 100 copies/mL or 100 IU/mL) for 1 month (or another specified time) means that a viral load measurement taken at least 1 month (or at another specified time) after determination of the initial measured value is no higher than the initial value. In some embodiments, the patient does not receive interferon lambda therapy during the specified time. In some embodiments, the patient does not receive any anti-HEV treatment during the specified time.

In some embodiments, therapy as disclosed herein is continued for a period of time until HEV RNA levels are below 3 log HEV RNA copies/mL (below 1,000 copies/mL), or sometimes until HEV RNA levels are below 2 log HEV RNA copies/mL (below 100 copies/mL) or below the level of detection. In some cases therapy may be continued for a period of time (such as 1 to 3 months or longer) after viral load has dropped to acceptably low levels (e.g., undetectable levels). In some embodiments, therapy is continued until the HEV viral load is reduced to undetectable levels.

In some embodiments, therapy as disclosed herein is continued for a period of time until HEV RNA levels are below 3 log IU/mL (below 1,000 IU/mL), or sometimes until levels are below 2 log IU/mL (below 100 IU/mL) or below the level of detection. In some embodiments, therapy is continued for a period of time until HEV RNA levels are below 3 log IU/g tissue (below 1,000 IU/g tissue), or sometimes until levels are below 2 log IU/g tissue (below 100 IU/g tissue), or until levels are below the level of detection. In some cases therapy may be continued for a period of time (such as 1 to 3 months or longer) after viral load has dropped to acceptably low levels (e.g., undetectable levels). In some embodiments, therapy is continued until the viral load is reduced to undetectable levels.

In some embodiments, a patient treated according to the methods described herein exhibits a reduction in HEV viral load to undetectable levels during the course of treatment, and the patient maintains the reduction in HEV viral load to undetectable levels for at least 12 weeks after the end of treatment. In some embodiments, a patient treated according to the methods described herein exhibits a reduction in HEV viral load to undetectable levels during the course of treatment, and the patient maintains the reduction in HEV viral load to undetectable levels for at least 24 weeks after the end of treatment.

In some embodiments, a patient treated according to the methods described herein exhibits an improvement in one or more liver function parameters. In some embodiments, the improved liver function is an improvement in one or more serum markers (e.g., one, two, three, four, five, six or more markers), such as serum albumin, bilirubin, alanine aminotransferase (ALT), aspartate aminotransferase (AST), prothrombin, alfa2-macroglobulin, apolipoproteinA1, haptoglobin, gamma-glutamyl transpeptidase (GGT). In some embodiments, a patient treated according to the methods described herein exhibits an improvement in liver fibrosis (e.g., as assessed by biopsy with histological analysis, transient ultrasound elastography (e.g., FibroScan), or magnetic resonance elastography). In some embodiments, treatment results in an improvement of at least 10%, at least 20%, at least 30%, at least 40%, at least 50% or more in one or more liver function parameters (e.g., an improvement in serum marker(s) or an improvement in liver fibrosis) in the patient as compared to prior to the onset of treatment. In some embodiments, treatment results in an improvement in one or more liver function parameters (e.g., an improvement in serum marker(s) or an improvement in liver fibrosis) to the level of a healthy control subject that is not infected with HEV. In some embodiments, the patient exhibits an improvement in serum ALT levels to a level that is within the upper limit of normal.

### Dose Escalation and Dose Reduction

In some embodiments, a patient being treated for HEV infection receives an adjustment in the dosing regimen of the interferon lambda therapy during the course of treatment. In some embodiments, the patient receives an escalating dosage regimen of interferon lambda, in that one or more later doses is a higher dose than one or more earlier doses. In some embodiments, an escalating dosage regimen may increase the patient's tolerance to the drug and minimize side effects. In some embodiments, dose escalation comprises administering interferon lambda at a first dose for a first treatment period followed by administering interferon lambda at a second dose for a second treatment period, wherein the second dose is larger than the first dose. In some embodiments, the length of time for the first treatment period is the same as the length of time for the second treatment period. In some embodiments, the first treatment period and the second treatment period are different lengths of time. In some embodiments, dose escalation further comprises administering one or more additional doses of interferon lambda for one or more additional treatment periods.

In some embodiments, the patient receives a dose reduction of interferon lambda, in that one or more later doses is a lower dose than one or more earlier doses. In some embodiments, dose reduction is prescribed if the patient exhibits unacceptable side effects. In some embodiments, the interferon lambda therapy comprises administering interferon lambda at a first dose for a first treatment period followed by administering interferon lambda at a second dose for a second treatment period, wherein the second dose is smaller than the first dose. For example, the first dose may be 60 μg/kg, and the second dose may be 30 μg/kg. In another example, the first dose may be from 250 μg/kg to 350 μg/kg (e.g., 300 μg/kg), and the second dose may be from 50 μg/kg to 250 μg/kg (e.g., 200 μg/kg). In some embodiments, the length of time for the first treatment period is the same as the length of time for the second treatment period. In some embodiments, the first treatment period and the second treatment period are different lengths of time.

### Formulation and Administration

Interferon lambda may be formulated for administration by any therapeutically appropriate route. In some embodiments, interferon lambda is formulated for administration by intravenous or subcutaneous administration. Other routes suitable for drug delivery, including systemic and localized routes of administration may be used.

In certain embodiments, interferon lambda is administered by subcutaneous injection, including but not limited to injection in the thigh or abdomen. The invention provides pharmaceutical formulations in which interferon lambda can be formulated into preparations for injection in accordance with the invention by dissolving, suspending or emulsifying it in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Unit dosage forms for injection or intravenous administration may comprise in a composition as a solution in sterile water, normal saline or another pharmaceutically acceptable carrier. Appropriate amounts of the active pharmaceutical ingredient for unit dose forms of interferon lambda are provided herein.

In some embodiments, interferon lambda (e.g., an interferon lambda 1 such as interferon lambda 1a) or an analog thereof is formulated and/or administered and/or modified as described in one of the following patent publications, incorporated by reference herein: US 2002/0039763, US 2004/0146988, US 2005/0014228, US 2005/0106680, US 2005/0106682, US 2007/0042468, US 2007/0048841, US 2007/0043209, US 2007/0049736, US 2008/0102490, US 2008/0214788, US 2009/0069274, US 2009/0326204, US 2010/0222266, US 2011/0054148, US 2011/0172170, US 2012/0036590, and US 2012/0258458.

### Antiviral Nucleotide or Nucleoside Analog Co-Therapy

In some embodiments, interferon lambda is used in combination with an (e.g., one or more) antiviral nucleotide or nucleoside analog to treat HEV infection or to reduce HEV viral load. A nucleoside analog is a nucleoside that contains a nitrogenous base analog and a sugar. A nucleotide analog is a nucleotide that contains a nitrogenous base analog, a sugar, and a phosphate group with one to three phosphates. Nitrogenous base analogs are structurally similar to naturally occurring nitrogenous bases found in DNA or RNA. Nucleotide and nucleoside analogs can be used in therapeutic drugs, including a range of antiviral products used to prevent viral replication in infected cells.

Antiviral nucleotide or nucleoside analogs that may be used in combination with the interferon lambda co-therapy described herein includes, but is not limited to, ribavirin (such as Rebetol® or Copegus®).

The dose of antiviral nucleotide or nucleoside analog administered to the patient is not particularly limited. In some embodiments, the antiviral nucleotide or nucleoside analog may be administered at a dose from 10 mg/kg to 50 mg/kg, e.g., 15 mg/kg to 50 mg/kg, 20 mg/kg to 50 mg/kg, 25 mg/kg to 50 mg/kg, 30 mg/kg to 50 mg/kg, 10 mg/kg to 45 mg/kg, 15 mg/kg to 45 mg/kg, 20 mg/kg to 45 mg/kg, 25 mg/kg to 45 mg/kg, 30 mg/kg to 45 mg/kg, 10 mg/kg to 40 mg/kg, 15 mg/kg to 40 mg/kg, 20 mg/kg to 40 mg/kg, 25 mg/kg to 40 mg/kg, 30 mg/kg to 40 mg/kg, 10 mg/kg to 35 mg/kg, 15 mg/kg to 35 mg/kg, 20 mg/kg to 35 mg/kg, 25 mg/kg to 35 mg/kg, 30 mg/kg to 35 mg/kg.

In some embodiments, the antiviral nucleoside or nucleotide analog is ribavirin. Said another way, interferon lambda may be used in combination with ribavirin. In one embodiment, for example, the treatment further comprises administering ribavirin at a dose of 25 mg/kg. In some embodiments, the nucleotide or nucleoside analog is ribavirin and the administered daily dose is 29-1000 mg, such as 29 mg, 50 mg, 100 mg, 200, mg, 300 mg, 400 mg, 500 mg, 600 mg, 700 mg, 800 mg, 900 mg, 1000 mg, and/or 29 mg to 100 mg, 29 mg to 400 mg, 200 mg to 600 mg, 500 mg to 800 mg, 600 mg to 900 mg, 700 mg to 1000 mg, as well as intermediate doses. In one embodiment, for example, ribavirin is administered at a daily dose of 80 mg. See, e.g., EASL Clinical Practice Guidelines on hepatitis E virus infection, J Hepatology, 68(6):1256-1271, June 2018, (doi: 10.1016/j.jhep.2018.03.005).

### III. EXEMPLARY EMBODIMENTS

As used below, any reference to a series of embodiments is to be understood as a reference to each of those embodiments disjunctively (e.g., “Embodiments 1-4” is to be understood as “Embodiments 1, 2, 3, or 4”).
Embodiment 1 is a method of treating a hepatitis E virus (HEV) infection in a human patient, the method comprising administering the patient a therapeutically effective amount of interferon lambda.
Embodiment 2 is the method of any of the preceding embodiment(s), wherein the interferon lambda is pegylated.
Embodiment 3 is the method of embodiment(s) 2, wherein the interferon lambda is pegylated interferon lambda-1a.
Embodiment 4 is the method of any of the preceding embodiment(s), wherein the interferon lambda is administered at a dose from 10 μg/kg to 500 μg/kg.
Embodiment 5 is the method of embodiment(s) 4, wherein the interferon lambda is administered at a dose from 10 μg/kg to 50 μg/kg.
Embodiment 6 is the method of embodiment(s) 4, wherein the interferon lambda is administered at a dose from 100 μg/kg to 500 μg/kg.
Embodiment 7 is the method of embodiment(s) 4, wherein the interferon lambda is administered at a dose from 100 μg/kg to 350 μg/kg or 250 μg/kg to 350 μg/kg or 275 μg/kg to 325 μg/kg or In some instances, the interferon lambda may be administered at a dose of 300 μg/kg±24.4 μg/kg.
Embodiment 8 is the method of embodiment(s) 4, wherein the interferon lambda is administered at a dose of 30 μg/kg.
Embodiment 9 is the method of any of the preceding embodiment(s), wherein the interferon lambda is administered twice weekly.
Embodiment 10 is the method of any of the preceding embodiment(s), wherein the interferon lambda is subcutaneously or intraperitoneally administered.
Embodiment 11 is the method of embodiment 10, wherein the interferon lambda is subcutaneously administered.
Embodiment 12 is the method of any of the preceding embodiment(s), wherein the interferon lambda is administered for at least 4 weeks.
Embodiment 13 is the method of embodiment 12, wherein the interferon lambda is administered for 8 weeks.
Embodiment 14 is the method of any of the preceding embodiment(s), wherein the treatment results in an HEV viral load that is below 100 IU/g tissue.
Embodiment 15 is the method of any of the preceding embodiment(s), wherein the treatment results in an HEV viral load that is below the level of detection.
Embodiment 16 is the method of any of the preceding embodiment(s), wherein the treatment results in an at least 3 log reduction in HEV viral load.
Embodiment 17 is the method of any of the preceding embodiment(s), further comprising administering an antiviral nucleotide or nucleoside analog.
Embodiment 18 is the method of embodiment 17, wherein the antiviral nucleotide or nucleoside analog is administered at a dose from 10 mg/kg to 50 mg/kg.
Embodiment 19 is the method of embodiment(s) 17 or 18, wherein the antiviral nucleotide or nucleoside analog is ribavirin.
Embodiment 20 is the method of any of embodiment(s) 17-19, wherein the antiviral nucleotide or nucleoside analog is ribavirin, and wherein the ribavirin is administered at a dose of 25 mg/kg.

### IV. EXAMPLES

The following examples are provided to illustrate, but not to limit, the claimed invention.

### Example 1: Interferon Lambda Treatment of HEV Infection in Liver Humanized Mouse Model

The present inventors and others have developed a liver humanized mouse model that is susceptible to patient-derived clinical HEV strains, which allows the study of REV's biology and evaluation of antiviral treatment candidates. This model is described, e.g., in Sari et al., “Hepatitis E Virus Shows More Genomic Alterations in Cell Culture than In Vivo”, Pathogens, 2019. 8(4):255; van de Garde, et al., “Hepatitis E Virus (HEV) Genotype 3 Infection of Human Liver Chimeric Mice as a Model for Chronic HEV Infection”, J. Virol., 2016. 90(9): p. 4394-4401; Vanwolleghem et al., “Factors determining successful engraftment of hepatocytes and susceptibility to hepatitis B and C virus infection in uPA-SCID mice”, J. Hepatol., 2010. 53(3): p. 468-476; Sayed et al., “Study of hepatitis E virus infection of genotype 1 and 3 in mice with humanised liver”, Gut, 2017. 66(5): p. 920-929; Michailidis et al., “Expansion, in vivo-ex vivo cycling, and genetic manipulation of primary human hepatocytes”, Proc. Nat'l Acad. Sci. USA, 2020. 117(3): p. 1678-1688. In this model, HEV infections do not elicit a primary interferon stimulated gene (ISG) response, persist for the life span of the mouse host, and can be cleared by stimulating the innate immune response via pegIFN-α injections. The present inventors examined the potential of pegIFN-λ, as a new treatment candidate against chronic gt3 HEV infections using this model. The study is described herein.

1. Materials and Methods

Thawed cryopreserved primary human hepatocytes (PHH, BD Gentest, Lot:342, Corning, Corning, NY, USA) or HepG2 cells (ATCC) were seeded with a 6-well plate in growth medium containing Dulbecco's modified Eagle's medium (DMEM; Lonza, Basel, Switzerland) supplemented with 10% fetal bovine serum (Greiner Bio-one, Kremsmünster, Austria), 2 mM 1-glutamine (Lonza, Basel, Switzerland), 1% penicillin-streptomycin (Lonza, Basel, Switzerland), and 20 mM HEPES (Lonza, Basel, Switzerland). After overnight seeding, HepG2 cells were washed once with phosphate-buffered saline (PBS, Oxoid, Hampshire, UK). Human hepatocyte cultures were used immediately. Thereafter, medium was supplemented with 0.01 mg/ml pegIFN-α (Pegasys, Roche, Basal, Switzerland) or 0.1 mg/ml pegIFN-λ, (provided by Eiger BioPharmaceuticals, Palo Alto, CA, USA) or PBS (Oxoid, Hampshire, UK) for 1 to 5 hours. After incubation, cells were collected in QIAzol® (Qiagen, Hilden, Germany).

NOD.Cg-Prkdcscid Il2rgtm1Sug Tg(Alb-Plau)11-4/ShiJic mice (uPA-NOG) and NOD.Cg-Prkdcscid Il2rgtm1Sug Tg(Alb-UL23)7-2/ShiJic (TK-NOG) mouse embryos were provided by Dr. Suemizu, Central Institute for Experimental Animals, Kawasaki, Japan. These mice are described, for example, in Hasegawa et al., “The reconstituted ‘humanized liver’ in TK-NOG mice is mature and functional”, Biochem. Biophys. Res. Comm′n, 2011. 405(3): p. 405-410; and Suemizu et al., “Establishment of a humanized model of liver using NOD/Shi-scid IL2Rgnull mice”, Biochem. Biophys. Res. Comm′n, 2008. 377(1): p. 248-252. Mice were bred at the Central Animal Facility of the Erasmus Medical Centre (Animal Ethical Committee nr 141-12-11). Homozygous uPA^{+/+} or TK+ mice were anesthetized and transplanted with 0.5 to 2×10⁶ viable cryopreserved PHH from 3 donors (Lonza, Lot:9F3003, Basel, Switzerland, and BD Gentest, Lot:342 and Lot:345, Corning, NY, USA) via intrasplenic injection. The demographic data of the hepatocyte donors is summarized in Table 1, below. At days −7 and −5 before transplantation, TK+ mice received an intraperitoneal (ip) ganciclovir injection to initiate liver damage as described in Hasegawa et al. 2011.

TABLE 1
Demographic Data of Hepatocyte Donors

| Hepatocyte | | | |
|---|---|---|---|
| Donor | Gender | Age | Race |
| Donor 1 | Male | 2 years | Caucasian |
| Donor 2 | Female | 2 years | Caucasian |
| Donor 3 | Female | 7 months | Caucasian |

Hepatocyte engraftment was determined by quantifying human albumin levels (hALB) in mouse serum with ELISA (Bethyl laboratories, Montgomery, TX, USA). Successfully engrafted mice (as defined by hALB>100 ug/ml) were intravenously inoculated with at least 10⁶ genomic element (GE) of the cell-culture adapted HEV gt3a Kernow C1/p6 strain (Dr. Feng, Nationwide Children's Hospital, Center for Vaccines and Immunity), 10⁶ IU of a patient-derived fecal HEV gt3c strain (GenBank accession number ORF1:JQ015423, ORF2:KP895854; as described in van de Garde et al., “Interferon-alpha treatment rapidly clears Hepatitis E virus infection in humanized mice”, Sci. Rep., 2017. 7(1): 8267), or left non-infected as a negative control. After viral inoculation, mice were housed individually.

HEV infected and non-infected mice were treated with pegIFN-α (0.03 mg/kg unless stated otherwise) twice weekly (every 3-4 days) via subcutaneous (sc) injection. Non-treated HEV infected and non-infected animals were used as controls. PegIFN-λ, was administered at dosages up to 0.3 mg/kg twice weekly (every 3-4 days) for up to 8 weeks, via sc or ip injection. Ribavirin (RBV) (Sandoz Biopharmaceuticals, Holzkirchen, Germany) was ip administered once daily in a volume of 200 μl at 25 mg/kg. Body weight and the assessment of clinical symptoms were determined 2-3 times a week. The evaluation details for the assessment of clinical symptoms is summarized in Table 2, below. Mouse liver and bile samples were collected at euthanasia. A liver fragment was stored into RNAlater® RNA Stabilization Reagent at −80° C. (Qiagen, Hilden, Germany). For some animals, weekly mouse fecal samples were obtained. HEV RNA levels were determined using an IS015189:2012-validated, internally controlled qRT-PCR.

TABLE 2
Evaluation details of animal behavior and appearances

| Clinical Score | Observation |
|---|---|
| CS 0 | Normal behavior, active, no aberrant fur |
| CS 0.5 | Pilo-erection AND/OR mild ruffled fur |
| CS 1 | Mild hunched posture OR mild ruffled |
| | fur AND slightly less active |
| CS 2 | Hunched posture AND ruffled fur AND less active |
| CS 3 | Hunched posture AND ruffled fur AND inactive |
| | (very low or absent mobility) |
| CS 4 | Death or killing request |

RNA was phenol-chloroform extracted from less than 30 mg liver tissue or cell lysates. cDNA was prepared with PrimeScript reverse transcriptase master mix (Takara Bio Inc., Kusatsu, Japan) according to manufacturer's protocol. Primer sets (using SYBR Green Master Mix (Thermo Fisher Scientific, Waltham, MA, USA)) and TaqMan primer-probes for mRNA expression analysis are listed in Table 3A and Table 3B, below. GAPDH was used as housekeeping gene control. Relative expression levels were calculated using the 2\hat{ }-dCt conversion. Cross reactivity of primers was checked using C57BL/6 and non-transplanted NOG mouse liver samples.

TABLE 3A
Target genes and Taqman probe IDs used for qPCR analysis

| Target Gene | Taqman Probe | Target Gene | Tagman Probe |
|---|---|---|---|
| CXCL10 | Hs01124251_g1 | MX1 | Hs00895608_m1 |
| DDX58 | Hs01061436_m1 | OAS1 | Hs00973637_m1 |
| IFIT1 | Hs01911452_s1 | RSAD2 | Hs00369813_m1 |
| IFNLR1 (IL28RA) | Hs00417120_m1 | STAT1 | Hs01013996_m1 |
| ISG15 | Hs01921425_s1 | TLR3 | Hs01551078_m1 |
| IFN-21 | Hs00601677_g1 | GAPDH | Hs00266705_g1 |

TABLE 3B
Custom designed primer sequences used for qPCR
analysis.

| Target | | SEQ |
|---|---|---|
| Gene | Sequence | ID NO |
| IFN-λ2F | 5′-GCTGAAGGACTGCAGGTGCCA-3′ | 1 |
| IFN-λ2R | 5′-GGGCTGGTCCAAGACGTCCA-3′ | 2 |
| IFN-λ-3F | 5′-GCTGAAGGACTGCAAGTGCCG-3′ | 3 |

F: forward primer; R: reverse primer

Human serum CXCL10 cytokine was measured in mouse serum samples using the Human CXCL10/IP10 Quantikine ELISA Kit (R&D Systems, Minneapolis, MN, USA) according to manufacturer's protocol.

Mouse livers were fixed in 4% formaldehyde (Merck Millipore, Burlington, MA, USA). For microscopy imaging, 4-5 μm cuts were prepared from paraffin embedded blocks. Immunofluorescent stainings of the livers were performed using a rabbit anti-ORF2 and a goat anti-human albumin (A80-229A, Bethyl Laboratories, Montgomery, TX, USA) antibodies followed by Alexa Fluor® 488 or 594-conjugated secondary antibodies (Thermo Fisher Scientific, Waltham, MA, USA). Nuclei were counterstained with DAPI (Thermo Fisher Scientific, Waltham, MA, USA). Slides were viewed with an EVOS® fluorescence microscope (Thermo Fisher Scientific, Waltham, MA, USA).

GraphPad Prism version 5.00 for Windows (GraphPad Software, San Diego, CA, USA) was used for statistical analysis. Unpaired Student t tests were used to obtain P values between groups and p<0.05 was accepted as statistically significant. *p<0.05, **p<0.01, ***p<0.001.

2. Results

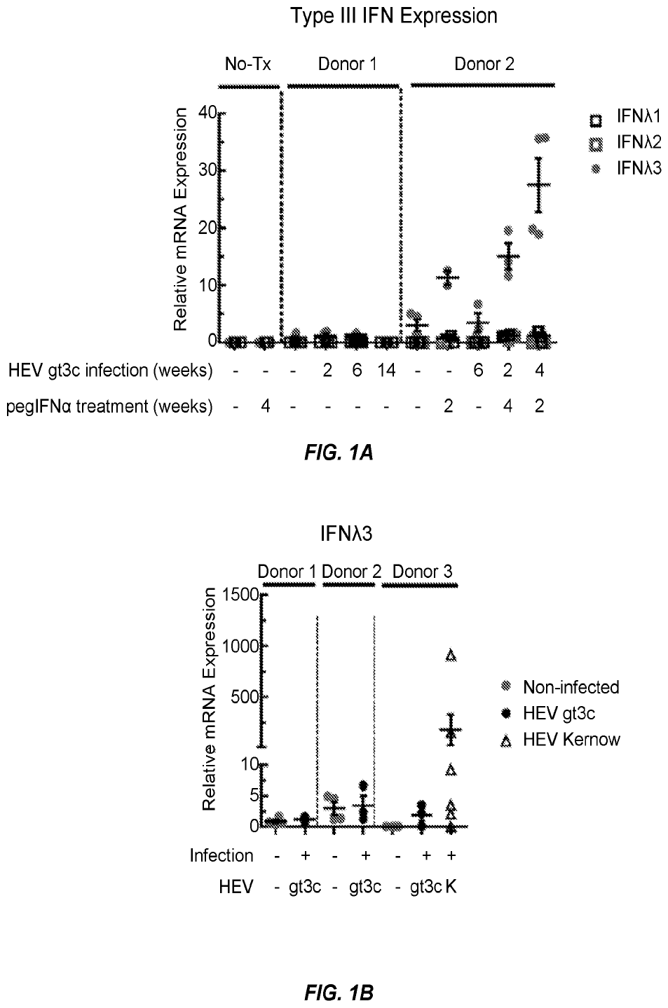

HEV Kernow virions can replicate in hepG2 cells or PHH despite a predominant type III IFN response with upregulated multiple ISGs. See Yin et al., Hepatitis E virus persists in the presence of a type III interferon response, PLoS Pathog, 2017. 13(5): e1006417. Previously, no ISG induction in liver humanized mice infected with different clinical HEV gt3 strains had been found. See van de Garde et al., 2017. In the study described herein, the present inventors examined endogenous type III IFN mRNA induction in vivo in HEV gt3c infected liver humanized mice. PegIFN-α-treated animals served as exogenously stimulated controls. Group size, infection duration and treatment details are shown in Table 4, below. As shown in FIG. 1A, a clinical HEV gt3c strain did not induce human type III interferons IFN-λ1, IFN-λ2, or IFN-λ3 mRNA levels in chimeric liver fragments despite ongoing replication for up to 14 weeks. A specific IFN-λ-3 mRNA expression was only observed following pegIFN-α treatment.

TABLE 4
Infection, treatment, virology, serology and sample size data of FIGS. 1A and 1B.

| | | Human | | | | | |
|---|---|---|---|---|---|---|---|
| | | albumin level | | | | Liver HEV RNA | |
| | | at sacrifice | Duration of | | | at sacrifice | |
| PHH | HEV | (μg/ml; | Infection | Treatment & | Duration of | (log IU/gr tissue, | Sample Size |
| Donor | Infection | mean ± SEM) | (pre-treatment) | Dosage | Treatment | GEM) | (mice/group) |
| Donor 1 | na | 4398 ± 1298.8 | na | na | na | na | 4 |
| | HEV gt3c | 739 ± 96.1 | 2 weeks | na | na | 5.68 | 4 |
| | | 1231 ± 595.5 | 6 weeks | | | 6.08 | 7 |
| | | 522 ± 298 | 14 weeks | | | 4.87 | 4 |
| Donor 2 | na | 3430.5 ± 1301.6 | na | na | na | na | 4 |
| | na | 666 ± 396.3 | na | pegIFN-α | 2 weeks | na | 2 |
| | | | | 30 μg/kg | | | |
| | HEV gt3c | 1489 ± 1047.4 | 6 weeks | na | na | 5.82 | 3 |
| | | 3520 ± 1579.3 | 2 weeks | pegIFN-α | 4 weeks | na | 3 |
| | | 1338 ± 409.6 | 4 weeks | 30 μg/kg | 2 weeks | na | 4 |
| Donor 3 | na | 378.5 ± 254.9 | na | na | na | na | 4 |
| | HEV gt3c | 770.2 ± 187.8 | 6 weeks | | | 3.80 | 3 |
| | HEV | 1357 ± 1556.2 | 6 weeks | | | 6.44 | 6 |
| | Kernow | | | | | | |
| na | na | na | na | na | na | na | 3 |
| na | na | na | na | pegIFN-α | 4 weeks | na | 2 |
| | | | | 30 μg/kg | | | |

GEM: Geometric Mean;
na: not applicable

Several experimental factors, such as the matrix surrounding PHEI and the multiplicity of infection, but also the viral strain might contribute to the higher ISG response found in HEV-infected hepG2 cell culture. To analyze the effect of the latter, the present inventors studied liver samples of liver humanized mice infected with either a clinical HEVgt3c strain or the cell culture adapted HEVgt3a Kernow strain for 6 weeks and results compared to the untransplanted and uninfected controls. As seen in FIG. 1B, mRNA expression was found to be induced only in the liver samples infected by the Kernow strain. No IFN-λ1 or IFN-λ2 mRNA expression was detected following infection with either HEV strains (data not shown).

In conclusion, the present inventors determined that IFN-λ-3 induction is HEV strain-specific and that the clinical HEV gt3c strain that apply in the studies has no stimulatory effect on IFN-λ, mRNA expression in vivo. Furthermore, the present inventors determined that IFN-λ, induction can be achieved via pegIFN-α treatment irrespective of an HEV infection.

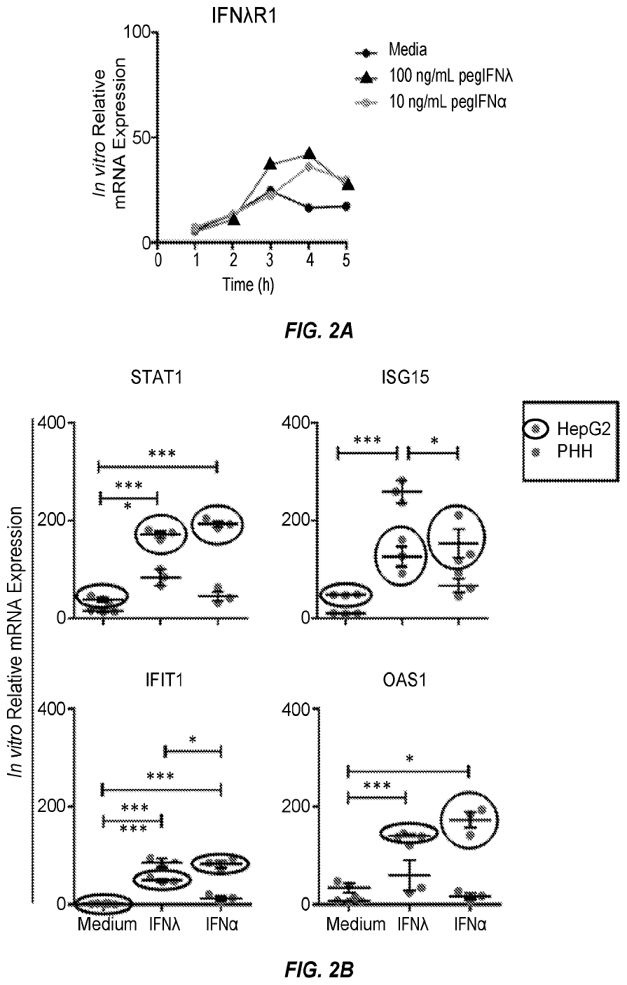

HEV clearance from liver humanized mice by pegIFN-α treatment is conventionally understood to be dependent on a robust ISG induction. See van de Garde et al., 2017. To examine whether pegIFN-λ, can result in a similar ISG induction as pegIFN-α, the present inventors studied the IFN-λR expression in PHH at baseline and upon stimulation with both cytokines. As seen in FIG. 2A, IFN-λR expression levels (IFNLR1 mRNA) increased following pegIFN-λ, and pegIFN-α treatment. The present inventors also examined the in vitro ISG response to exogenous IFN-α and IFN-λ. In previous studies with B cells, it was shown that IFN-λ, is 10 times less potent than IFN-α in vitro. See de Groen et al., “IFN-lambda is able to augment TLR-mediated activation and subsequent function of primary human B cells”, J. Leukocyte Biology, 2015, 98(4): p. 623-630. The present inventors therefore incubated primary human hepatocytes or hepatoma cells (HepG2 cells) with 0.1 mg/mL pegIFN-λ, and 0.01 mg/mL pegIFN-α respectively and determined the change in mRNA expression levels of ISGs: STAT1, ISG15, IFIT1, and OAS1. ISG induction in hepG2 cells was comparable for both cytokines at 10-fold lower doses for IFN-α, as shown in FIG. 2B. PHH were less sensitive to this 10-fold lower exogenous pegIFN-α with numerically higher ISG responses for pegIFN-λ, which reached statistical significance for IFIT1 and ISG15 (*p<0.05, **p<0.01, ***p<0.001). Both exogenous pegIFN-α and pegIFN-λ, were potent inducers of ISG in PHH and HepG2 cells.

In conclusion, the present inventors determined that exogenous IFN-λ, leads to an increase of both the IFN-λR expression levels and downstream ISG in primary human hepatocytes.

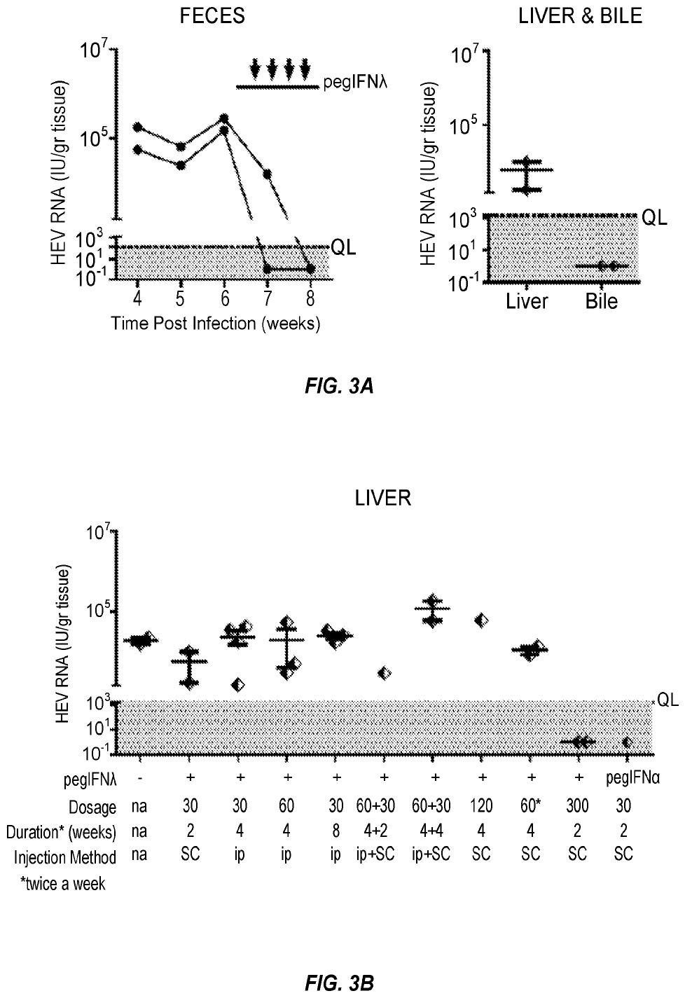

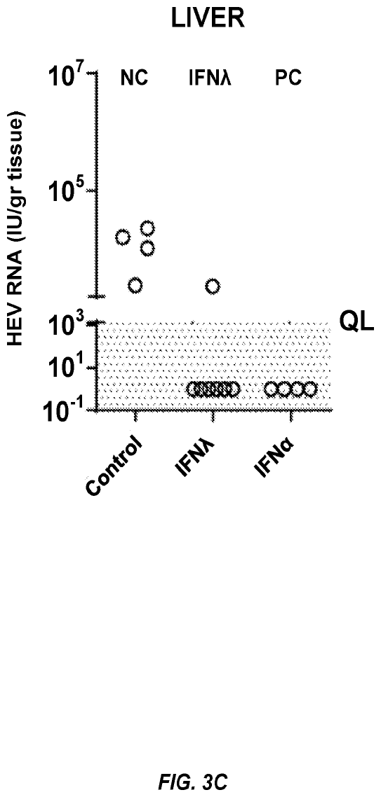

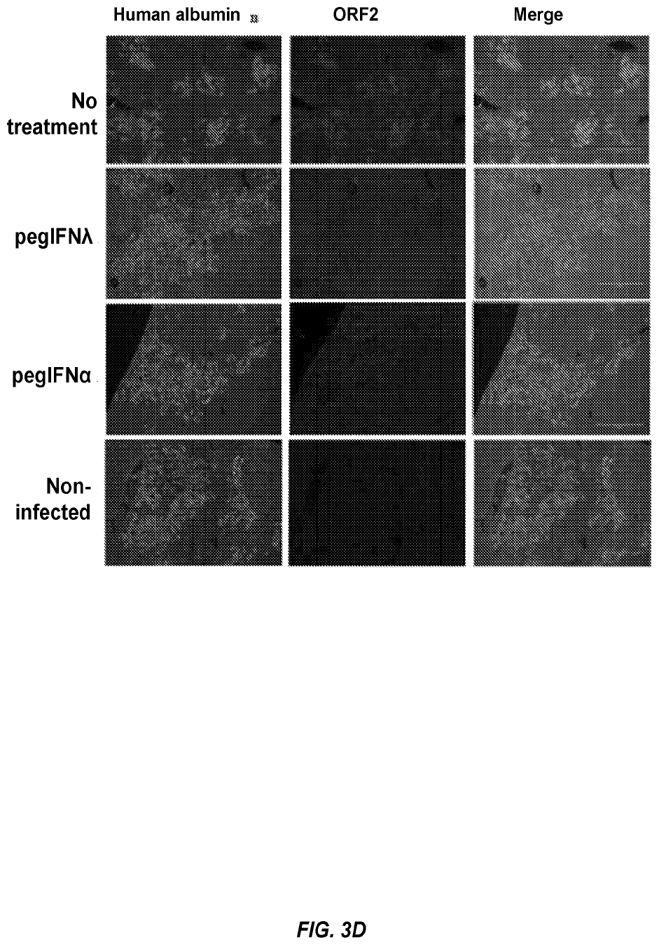

Given the in vitro induction of ISG in both hepG2 and PHH by IFN-λ, the present inventors examined whether pegIFN-λ, was able to clear a HEV gt3 infection in vivo in liver humanized mice. First, 0.03 mg/kg pegIFN-λ, was applied twice weekly for 2 weeks. As shown in FIG. 3A, HEV fecal shedding dropped after 2 doses of pegIFN-λ. However, bile and liver samples collected at sacrifice after 4 injections (i.e., after 2 weeks) showed a sterilization in bile, but HEV RNA persistence in liver fragments. HEV fecal shedding is thus sensitive to pegIFN-λ, treatment, but fecal or bile sterilization data may not be reflective of liver clearance.

Given the delayed kinetics of ISG induction by IFN-λ, compared to IFN-α, the present inventors examined different pegIFN-λ, doses and durations. The present inventors specifically analyzed liver samples to determine the optimal HEV clearance treatment. In addition, daily RBV treatment for 12 days was added in one cohort, to examine synergistic antiviral effects of this established HEV antiviral. These results are shown in FIG. 3B and Table 5 below. Non-treated and pegIFN-α-treated chimeric mice served as negative and positive controls respectively. All applied doses of pegIFN-λ. (up to 0.3 mg/kg) and durations (up to 8 weeks) were well tolerated by both infected and non-infected mice, without weight loss or behavioral changes.

TABLE 5
Treatment regimen, virology and sample size data for FIGS. 3B and 3C.

| Pre-treatment | | | | | |
|---|---|---|---|---|---|
| feces | | | | | Liver HEV |
| HEV RNA | | | No. of | | RNA titer |
| titer | | | injections | | (log IU/gr |
| (log IU/gr | | | (twice | Admin. | tissue, |
| tissue, GEM) | Treatment | Dosage | weekly) | Route | GEM) |
| 4.15 | na | na | na | na | 3.80 (n = 3) |
| 5.80 | pegIFN-λ | 30 μg/kg | 4, 8 or 16 | sc or ip | 4.31 (n = 9) |
| 5.58 | pegIFN-λ | 60 μg/kg | 8 | ip | 4.47 (n = 3) |
| nd | pegIFN-λ | 60 μg/kg + | 8 + 4 | ip + SC | 3.48 (n = 1) |
| | | 30 μg/kg | | | |
| nd | pegIFN-λ | 60 μg/kg + | 8 + 8 | ip + SC | 5.08 (n = 2) |
| | | 30 μg/kg | | | |
| 4.18 | pegIFN-λ | 60 μg/kg + | 8 | SC + ip | 4.05 (n = 2) |
| | | 25 mg/kg | RBV: | | |
| | | RBV | 12 days/ | | |
| | | | daily | | |
| 3.99 | pegIFN-λ | 120 μg/kg | 8 | SC | 4.79 (n = 1) |
| 4.10 | pegIFN-λ | 300 μg/kg | 4 | SC | undetectable |
| | | | | | (8/9) |
| | | | | | 3.35 (1/9) |
| 4.37 | pegIFN-α | 30 μg/kg | 4 | SC | undetectable |
| | | | | | (n = 5) |

GEM: Geometric Mean; na: not applicable; nd: not done

As seen in the FIG. 3B and Table 5, a complete sterilization of HEV liver titers required 4 twice weekly injections of pegIFN-λ, at a dose of 0.3 mg/kg (n=2). In an independent confirmatory experiment, HEV clearance was observed in 8 out of 9 mice treated with 0.3 mg/kg for 2 weeks, while complete sterilization was seen in positive controls treated with 0.03 mg/kg pegIFN-α (n=4) and persistent infection in untreated controls (n=4) (FIG. 3C). Confocal images of liver slides showed no HEV ORF2 positive human clusters in pegIFN-λ, and pegIFN-α treated animals, while human albumin expressing islands in liver fragments of non-treated controls remained HEV ORF2 positive (FIG. 3D).

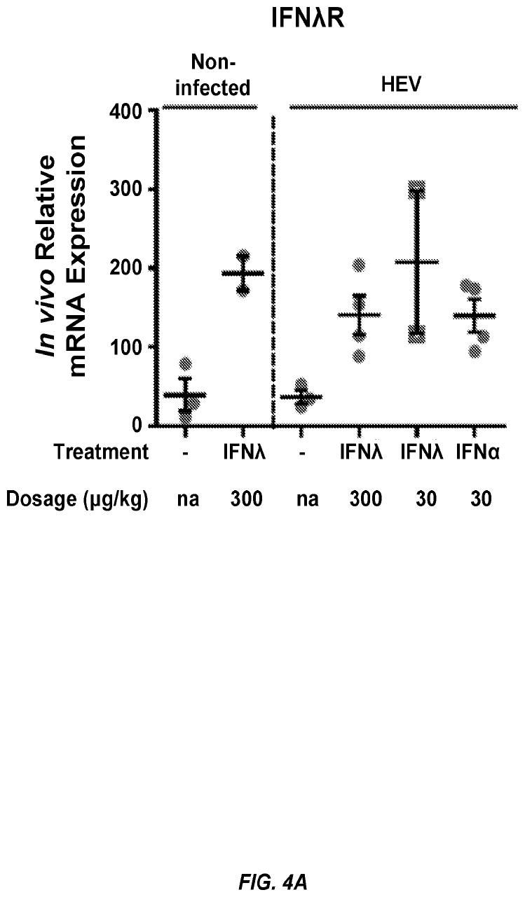

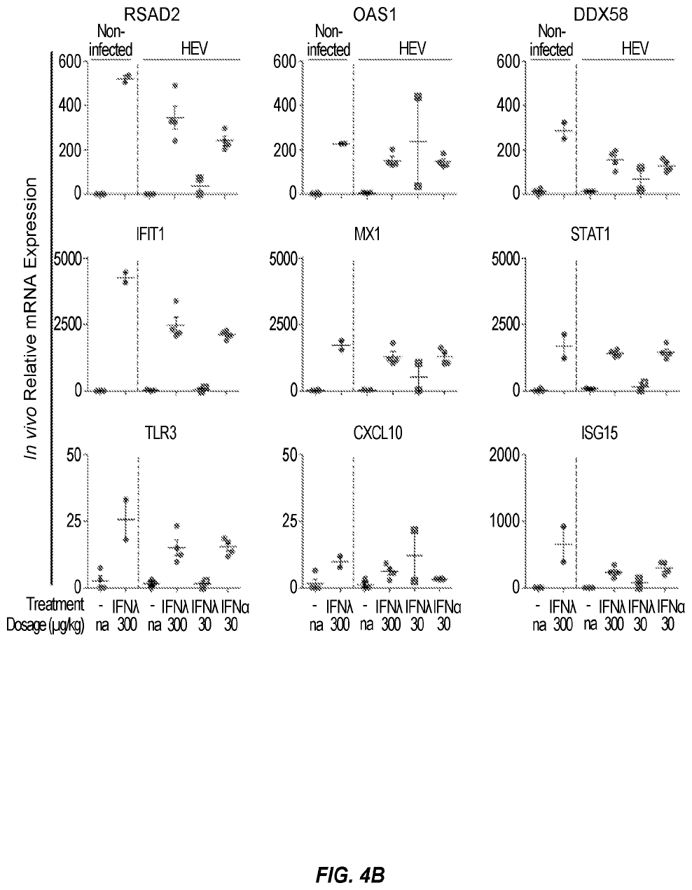

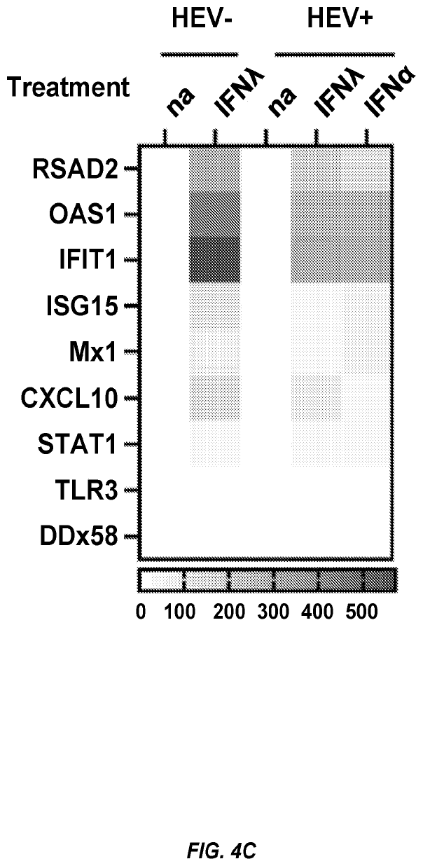

To evaluate whether this higher pegIFN-λ, dose requirement is reflected in ISG induction in vivo, the present inventors assessed the stability of the IFN-λ.R1 mRNA expression levels in livers of humanized mice treated with the optimal antiviral doses of 0.3 mg/kg pegIFN-λ., 0.03 mg/kg pegIFN-λ, and pegIFN-α. Compared to untreated infected animals, the IFN-λR expression was induced irrespective of the applied cytokine or dose (FIG. 4A). However, the expression levels of 9 different human-specific ISG were not significantly different in 0.3 mg/kg pegIFN-λ, and 0.03 mg/kg pegIFN-α treatment conditions (FIG. 4B). Moreover, ISG induction but not IFN-λR1 mRNA expression for the 0.03 mg/kg pegIFN-λ, treatment was lower than observed for both 0.3 mg/kg pegIFN-λ and 0.03 mg/kg pegIFN-α treatments. These data support a lower biological activity of IFN-λ vs IFN-α. However, HEV gt3 infection did not induce an intrahepatic ISG, nor a type III IFN response (FIG. 4B; data not shown). Nevertheless, the applied pegIFN-λ, treatment strongly induced intrahepatic ISG mRNA expression, with a comparable profile to that of pegIFN-α, as shown in FIG. 4C.

In conclusion, the present inventors determined that HEV gt3 in vivo antiviral efficacy of equimolar pegIFN-λ, is lower than pegIFN-α. A 10-fold higher pegIFN-λ, dose is required to reach a similar intrahepatic ISG induction and an 88.9% HEV clearance rate (8/9 mice). Treatment with peg-IFN-λ, leads to intrahepatic ISG induction, loss of HEV ORF2 protein expression in human albumin positive clusters, and HEV RNA liver clearance in liver-humanized mice. The here tested optimal mouse dose would be equivalent to a human pegIFN-λ, dose of ±24.4 μg/kg.

3. Summary

In the present study, HEV infections resulted in a minimal IFN-λ, induction that was strain-dependent. Exogenous IFN-λ stimulated ISG mRNA expression in primary human hepatocytes and hepatoma cell line in vitro. Compared to IFN-α, 10-fold higher doses of pegIFN-λ are required to reach a similar intrahepatic ISG induction and HEV clearance. Nevertheless, these higher doses were well tolerated by both HEV infected and non-infected mice.

In HCV infections, non-response to exogenous IFN-α has previously been ascribed to high baseline levels of intrahepatic ISG and IFN-λR expression levels. See Duong et al., “IFN-lambda receptor 1 expression is induced in chronic hepatitis C and correlates with the IFN-lambda3 genotype and with nonresponsiveness to IFN-alpha therapies”, J. Exp. Med., 2014. 211(5): p. 857-868. A similar mechanism has been found in vitro in HEV-infected hepG2 cells, when high JAK-STAT signaling due to a type III IFN response precluded further stimulation by exogenous IFN-λ. See Yin et al., “Hepatitis E virus persists in the presence of a type III interferon response”, PLoS Pathog, 2017. 13(5): e1006417. By the above-described study, the present inventors have found that type III IFN induction was limited to animals infected with the cell culture-adapted HEV gt3 Kernow strain, which persisted in the presence of IFN-λ-3 expression for 6 weeks. These data confirm the previous in vitro type III IFN induction upon HEV Kernow infection. The reason why no ISG induction was found in animals infected with a HEV gt3c clinical strain belonging to the same Glade as the Kernow strain is currently unclear, but might be representative for clinical HEV infections. Indeed, unlike fully sequenced clinical HEV strains, an insertion of a human S17 ribosomal fragment increases the in vitro fitness of the Kernow strain. This is a characteristic that may be transferred to other strains like the HEV1 Sar-55. This additional ribosomal protein S17 fragment might alter the intracellular viral sensing machinery and result in differences in ISG and type III interferon induction between Kernow and clinical HEV strain.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

What is claimed:

1. A method of treating a hepatitis E virus (HEV) infection in a human patient, the method comprising administering the patient a therapeutically effective amount of interferon lambda.

2. The method of claim 1, wherein the interferon lambda is pegylated.

3. The method of claim 2, wherein the interferon lambda is pegylated interferon lambda-1a.

4. The method of any one of claims 1-3, wherein the interferon lambda is administered at a dose from 10 μg/kg to 500 μg/kg.

5. The method of claim 4, wherein the interferon lambda is administered at a dose from 10 μg/kg to 50 μg/kg.

6. The method of claim 4, wherein the interferon lambda is administered at a dose from 100 μg/kg to 500 μg/kg.

7. The method of claim 4, wherein the interferon lambda is administered at a dose from 100 μg/kg to 350 μg/kg or 250 μg/kg to 350 μg/kg or 275 μg/kg to 325 μg/kg.

8. The method of claim 4, wherein the interferon lambda is administered at a dose of 30 μg/kg.

9. The method of any one of claims 1-3, wherein the interferon lambda is administered twice weekly.

10. The method of any one of claims 1-3, wherein the interferon lambda is subcutaneously or intraperitoneally administered.

11. The method of claim 10, wherein the interferon lambda is subcutaneously administered.

12. The method of any one of claims 1-3, wherein the interferon lambda is administered for at least 4 weeks.

13. The method of claim 12, wherein the interferon lambda is administered for 8 weeks.

14. The method of any one of claims 1-3, wherein the treatment results in an HEV viral load that is below 100 IU/g tissue.

15. The method of any one of claims 1-3, wherein the treatment results in an HEV viral load that is below the level of detection.

16. The method of any one of claims 1-3, wherein the treatment results in an at least 3 log reduction in HEV viral load.

17. The method of any one of claims 1-3, further comprising administering an antiviral nucleotide or nucleoside analog.

18. The method of claim 17, wherein the antiviral nucleotide or nucleoside analog is administered at a dose from 10 mg/kg to 50 mg/kg.

19. The method of claim 17 or 18, wherein the antiviral nucleotide or nucleoside analog is administered at a daily dose from 29 mg to 1000 mg.

20. The method of any of claims 17-19, wherein the antiviral nucleotide or nucleoside analog is ribavirin.

21. The method of any of claim 17-20, wherein the antiviral nucleotide or nucleoside analog is ribavirin, and wherein the ribavirin is administered at a dose of 25 mg/kg.


SEQUENCE LISTING (3 sequences)

SEQ ID NO: 1 - DNA, 21 bp, Artificial Sequence
  Description of Artificial Sequence Synthetic primer
gctgaaggactgcaggtgcca

SEQ ID NO: 2 - DNA, 20 bp, Artificial Sequence
  Description of Artificial Sequence Synthetic primer
gggctggtccaagacgtcca

SEQ ID NO: 3 - DNA, 21 bp, Artificial Sequence
  Description of Artificial Sequence Synthetic primer
gctgaaggactgcaagtgccg